(19) United States
(12) Patent Application Publication
(10) Pub. No.: US 2008/0275285 A1
(43) Pub. Date: Nov. 6, 2008

(54) Reduction of ethanol, aldols, polyols and polar organic compounds to hydrocarbons using natural gas

(75) Inventor: Melvin Keith Carter, Lincoln, CA (US)

Correspondence Address: Carter Technologies, 2300 Sutter View Lane, Lincoln, CA, 95648, US

(73) Assignee: Carter Technologies, Lincoln, CA (US)

(21) Appl. No.: 11/797,509

(22) Filed: May 4, 2007

(51) Int. Cl. C07C 4/06

(52) U.S. Cl. 585/648; 585/752

(57) ABSTRACT

Catalytic processes have been developed for reductive conversion of alcohols, aldehydes, ketones, carboxylic acids, esters, ethers, amines, thiols, phosphines and aldols to hydrocarbons using methane, natural gas or other gaseous hydrocarbons. Aliphatic hydrocarbons including propane, nonanes, tridecanes, gasoline, diesel fuel, oils, solvents and other organic compounds can be formed by this catalytic process. The catalysts are based on di-metal, tri-metal and/or poly-metal backbone or molecular string type compounds of transition metals, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold and combinations thereof in conjunction with a non-fluoride magnesium halide.

### BACKGROUND

1. Field of Invention

This invention relates to catalytic reduction of reducible compounds without the use of hydrogen gas, rather employing methane, natural gas and/or other gaseous hydrocarbons with catalysts based on molecular strings of di-, tri- and/or poly-groups of bonded transition metal complexes in conjunction with a magnesium salt comprising non-fluoride magnesium halides.

2. Description of Prior Art

Catalytic reduction of alcohols, aldehydes, ketones and other compounds to hydrocarbons has been conducted previously with the nearly exclusive use of hydrogen gas. Hydrogen gas is commonly manufactured from hydrocarbons such as methane with the loss of carbon or from carbon and water at high temperature by a steam reformation process. Production of hydrogen by these processes is expensive but may be less expensive than the electrolytic process. Natural gas and methane are available renewable resources, although natural gas is presently taken from underground wells.

Reaction of methane with other organic compounds has long been sought as a method of obviating the need for hydrogen gas but such reactions proved to be recalcitrant. Current industrial chemical methane processes generate synthesis gas, halocarbons, hydrogen cyanide, acetylene, carbon disulfide and carbon but reaction efficiencies for production of saturated hydrocarbons are quite low. A number of chemical reaction paths have previously been investigated for use of methane as a reactant including controlled oxidation of methane to alcohols and aldehydes, chlorination of methane to make reactive intermediates and application of methane sulfonic acid to produce methanated compounds. Controlled oxidation has produced a myriad of compounds including carbon dioxide, carbon monoxide, water and low concentrations of methanol, formaldehyde and resinous residues. Chlorination of methane has also been conducted, however formation of higher molecular weight hydrocarbons is conducted with formation of metal chlorides, hydrogen chloride or other chlorinated compounds resulting in a loss of chlorine, its acids or its salts. In the chemical industry, methane has been a raw material for the manufacture of methanol (CH₃OH), formaldehyde (CH₂O), nitromethane (CH₃NO₂), chloroform (CH₃Cl), carbon tetrachloride (CCl₄), and some freons. The reactions of methane with chlorine and fluorine are triggered by light. When exposed to bright visible light, mixtures of methane with chlorine or fluorine react explosively. Application of methane sulfonic acid as a viable reactant is of limited use and produces sulfuric acid as a by product. The aforementioned methane reaction routes are expensive, produce significant by products and hazardous waste residues.

Direct methanation of hydrocarbons has been sought but not previously accomplished. Reformation of organic compounds has been conducted in pressurized reactions at elevated temperatures in the presence of selected transition metal catalysts. Production of methane has been conducted converting hydrocarbon liquids on catalyst composed of group IVb, Vb, VIb & VIII metals at elevated temperatures to gases similar to methane gas as taught in U.S. Pat. No. 4,284,531, issued Aug. 18, 1981. This process generates methane like gases but does not teach use of the products for reduction. U.S. Pat. No. 4,086,261, issued Apr. 25, 1978, discloses hydrogenation of carbon oxides and other feed stocks forming methanol, other alcohols and similar products. Here again this process has limited use for reasons of economics.

The present application teaches use of methane as a direct reducing agent for polar compounds such as alcohols, aldehydes, ketones, carboxylic acids, esters, ethers, amines, thiols, phosphines and aldols resulting in formation of hydrocarbons using selected catalysts. For example, catalytic methane reduction or methanation of aldols, organic compounds possessing both alcohol and aldehyde groups, to hydrocarbons proceeds readily to hydrocarbons at elevated temperatures. Aldol compositions such as C₁₀H₂₀O₅ can be reduced in the presence of methane to form C₁₅H₃₀, C₁₆H₃₄ and water. The presence of residual aldehyde intermediates can be eliminated by control of reaction conditions.

The invention disclosed in this application is different from the classifications referenced above in that aliphatic hydrocarbons have been directly produced catalytically from alcohols, aldehydes and aldols using methane as the reactive reducing agent. The catalysts were prepared from di-metal, tri-metal and/or poly-metal backbone or molecular string type transition metal catalysts in conjunction with a non-fluorinated magnesium halide but without addition of aggressive chemical oxidizing agents and without addition of other strong chemicals. Liquid hydrocarbons were formed directly from liquid aldols by this methanation process.

It is an object of this invention, therefore, to provide a molecular string type transition metal catalytic process for methanation of polar compounds resulting in formation of aliphatic hydrocarbons.

It is another object of this invention to provide molecular string type transition metal catalysts in conjunction with a non-fluorinated magnesium halide for direct production of hydrocarbons including nonanes, endecanes, hydrocarbon oils, solvents, gasoline, jet fuel, diesel fuel, heating and lubricating oils, as well as other types of hydrocarbons.

Other objects of this invention will be apparent from the detailed description thereof which follows, and from the claims.

### SUMMARY OF THE INVENTION

This invention describes a chemical process for reduction of polar organic compounds forming aliphatic hydrocarbons using methane, natural gas or other gaseous hydrocarbons and using selected members of a family of transition metal catalysts, based on a di-metal, tri-metal and/or poly-metal backbone or string type compounds in conjunction with a non-fluorinated magnesium halide. These catalysts have been effectively demonstrated to be active for formation of many hydrocarbons.

### DETAILED DESCRIPTION OF THE INVENTION

The process for catalytic reduction of polar organic compounds comprising alcohols, aldehydes, ketones, carboxylic acids, esters, ethers, amines, thiols, phosphines and aldols using methane, natural gas or other gaseous hydrocarbons is a general process designed to replace hydrogen in production of hydrocarbon fuels. This process uses ethanol and products produced from ethanol, including aldehydes, aldols and polyols, as feed compounds reducing them to useful hydrocarbons including gasoline, diesel fuel, heating oils, lubricants, other hydrocarbon fuels and numerous other industrial organic compounds.

The process is based on catalysts possessing multiple metal type transition metal compounds, such as [iron]₂ or [manganese]₂ type compounds and numerous others in conjunction with non-fluorinated magnesium halides. These catalysts have been designed based on a formal theory of catalysis, and the catalysts have been produced, and tested to prove their activity. The theory of catalysis rests upon a requirement that a catalyst possess a linear backbone or molecular string such that transitions from one molecular electronic configuration to another be essentially barrier free so reactants may proceed freely to products. Catalysts effective for methanation of polar organic compounds in formation of hydrocarbons can be made from di-metal, tri-metal and/or poly-metal backbone or molecular string type compounds of the transition metals, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold and combinations thereof. These catalysts are made in the absence of oxygen so as to produce compounds in the divalent state or other low oxidation state. Anions employed for these catalysts comprise chloride, bromide, cyanide, isocyanate, thiocyanate, metal cyanides, sulfate, phosphate, oxide, acetate, oxalate and other more complex groups, only some of which are found to be non-toxic to the natural environment. Mixed transition metal compounds have also been found to be effective catalysts for methanation of polar organic compounds. These catalysts are effective with a non-fluorinated magnesium halide promoter.

Different first row transition metal catalysts have been prepared for methanation of ethanol, aldols, polyols and other polar organic compounds in producing hydrocarbons at modest pressures and at temperatures of 250° C. to 330° C. Ethanol reactant produced gaseous hydrocarbons while aldols produced liquid hydrocarbons. These same catalysts were also effective for production of solid hydrocarbons including waxy compounds. Reaction pressures of 0 to 60 psi have been employed in many of the catalytic methanation processes, although higher pressures are also effective. This process may also be employed for reduction of carbon oxides forming hydrocarbons.

### Catalyst Selection Considerations

The fundamentals of catalysis effort forms a basis for selecting molecular catalysts for specified chemical reactions through computational methods by means of the following six procedural steps. An acceptable methanation mechanism, involving a pair of metal atoms, was established for methane gas in the presence of a polar reactant (step 1). A specific transition metal, such as cobalt, was selected as a possible catalytic site as found in an M-M or Co—Co string (step 2), bonded with sets of polar organic molecules in symmetric configurations, and having a computed bonding energy to the associated polar reactants of less than −60 kcal/mol (step 3). The first valence state for which the energy values were two-fold degenerate was 2+ (step 4). Acetate, chloride and other anions may be chosen provided they are chemically compatible with the metal, M (Co), in formation of the catalyst (step 5). A test should also be conducted to establish compliance with the rule of 18 (or 32) to stabilize the catalyst so compatible ligands may be added to complete the coordination shell (step 6). This same process may be applied for selection of a catalyst using any of the first, second or third row transition metals, however, only those with acceptable low positive or negative bonding energies can produce effective catalysts. Approximate, computed, relative bonding energy values may be computed using a semi-empirical algorithm. This computational method indicated that several of the first row transition metal complexes can produce usable catalysts once the outer coordination shell has been completed with ligands. Second row and third row transition metal complexes were also indicated to produce active catalysts.

Transition metal catalysts loaded onto silica, silica-alumina, alumina or other support materials have been employed. Non-fluorinated magnesium halide compounds combined with and/or loaded onto the catalyst support were effective promoters of the catalytic process. Addition of 0.01 to 90 percent of a catalyst and a balance of non-fluorinated magnesium halide salts promoted methanation reduction reactions.

### Description of Catalyst Preparation

Catalyst preparation has been conducted using nitrogen saturated solvents and nitrogen blanketing to minimize or eliminate air oxidation of the transition metal compounds during preparation. Transition metal catalysts, effective for methanation of polar organic compounds, can be produced by combining transition metal salts in their lowest standard oxidation states. Thus, such transition metal catalysts can be made by mixing transition metal (I or II) chlorides with sodium acetate or ammonium hydrogen oxalate in a 1 to 2 or 1 to 3 ratio, or by forming transition metal compounds in a reduced state by similar means where di-, tri- and/or poly-metal compounds result.

### EXAMPLE 1

The cobalt acetate catalyst may be prepared in a nitrogen atmosphere by addition of 0.15 gram (2 mmol) of ammonium acetate to 0.25 gram (1 mmol) of light pink colored cobalt (II) acetate tetrahydrate dispersed in 15 grams of nitrogen purged ethanol with mixing and gentle heating. To the resulting deep magenta to purple solution was added to 20 grams of a silica alumina support and the mixture was dried under nitrogen producing the catalyst.

### EXAMPLE 2

Preparation of copper oxalate catalyst may be conducted in a nitrogen atmosphere by addition of 0.28 gram (2 mmol) of ammonium oxalate to 0.25 gram (1 mmol) of blue colored copper (II) sulfate pentahydrate dissolved in 10 grams of nitrogen purged water with mixing. To the resulting suspension that dissolved slowly was added 20 grams of silica alumina support and the mixture was dried under nitrogen producing the catalyst.

### EXAMPLE 3

Preparation of manganese oxalate catalyst may be conducted in a nitrogen atmosphere by addition of 0.28 gram (2 mmol) of ammonium oxalate to 0.20 gram (1 mmol) of manganese (II) chloride tetrahydrate dissolved in 10 grams of nitrogen purged water with mixing. To the resulting solution was added 20 grams of silica alumina support and the mixture was dried under nitrogen producing the catalyst.

### Catalytic Methanation Reactions

The solid cobalt catalyst of example 1 (˜20 grams) was mixed with approximately 2 grams of magnesium chloride and loaded into a one half inch diameter stainless steel reactor tube fit with reactant inlet, pressure and temperature monitoring, product outlet and a means of controlling methane flow rate. In addition, a means of dehydration of the reaction stream was applied. The reactor was flushed with methane and heated to 250° C. to start the reaction. Liquid aldols were injected into the reactor 50 to 100 microliters at a time until at least 0.5 milliliter had been added. Liquid hydrocarbon products were removed from the outlet and analyzed. This process was repeated several times for temperatures in the range of 250° C. to 330° C. and pressures in the range of 30 to 60 psi. This reaction process was repeated using cobalt oxalate under essentially the same conditions with similar results.

What is claimed:

1. A process for catalytic methanation of reactants comprising alcohols, aldehydes, ketones, carboxylic acids, esters, ethers, amines, thiols, phosphines and aldols to hydrocarbons using reducing agents comprising methane, natural gas or other gaseous hydrocarbon reducing agents, catalysts made from di-metal, tri-metal and/or poly-metal backbone or molecular string type compounds of transition metals, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold and combinations thereof in conjunction with a magnesium salt promoter comprising magnesium chloride, bromide or iodide.

2. A process for catalytic methanation of reactants comprising alcohols, aldehydes, ketones, carboxylic acids, esters, ethers, amines, thiols, phosphines and aldols to hydrocarbons using reducing agents comprising methane, natural gas or other gaseous hydrocarbon reducing agents, catalysts made from di-metal, tri-metal and/or poly-metal backbone or molecular string type compounds of transition metals, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold and combinations thereof in conjunction with a magnesium salt promoter comprising magnesium chloride, bromide or iodide at temperatures between 250° C. and 330° C.

3. A process for catalytic methanation of reactants comprising alcohols, aldehydes, ketones, carboxylic acids, esters, ethers, amines, thiols, phosphines and aldols to hydrocarbons using reducing agents comprising methane, natural gas or other gaseous hydrocarbon reducing agents, catalysts made from di-metal, tri-metal and/or poly-metal backbone or molecular string type compounds of transition metals, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold and combinations thereof in conjunction with a magnesium salt promoter comprising magnesium chloride, bromide or iodide at temperatures between 250° C. and 330° C., and pressures below 7 atmospheres.

4. A process for catalytic methanation of reactants comprising ethanol, aldehydes and aldols to hydrocarbons using reducing agents comprising methane, natural gas or other gaseous hydrocarbons reducing agent, catalysts made from di-metal, tri-metal and/or poly-metal backbone or molecular string type compounds of transition metals, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold and combinations thereof in conjunction with a magnesium salt promoter comprising magnesium chloride, bromide or iodide at temperatures between 250° C. and 330° C., and pressures below 7 atmospheres.
